(19) United States
(12) Patent Application Publication
(10) Pub. No.: US 2026/0174383 A1
(43) Pub. Date: Jun. 25, 2026

(54) WEARABLE DEVICE FOR SLEEP APPLICATIONS, AND SYSTEMS AND METHODS FOR OPERATION THEREOF

(71) Applicant: Drowsy Digital, Inc., Dover, DE (US)

(72) Inventors: Naganagouda PATIL, Westborough, MA (US); Brian MULCAHEY, Sudbury, MA (US); Pramodh ALATHUR, Bangalore (IN); Steven TORKU, Uxbridge, MA (US); Katie MOENKHAUS, Madison, NH (US); Charles TAYLOR, Millford, CT (US)

(21) Appl. No.: 19/540,332

(22) Filed: Feb. 13, 2026

Related U.S. Application Data
(63) Continuation of application No. PCT/US2024/042742, filed on Aug. 16, 2024.
(60) Provisional application No. 63/533,019, filed on Aug. 16, 2023.

(51) Int. Cl. A61B 5/00; A61B 5/08

(52) U.S. Cl. CPC A61B 5/4812; A61B 5/0816; A61B 5/7475

(57) ABSTRACT

Embodiments described herein relate to a wearable device for monitoring a user's sleep metrics and adjusting the user's sleep conditions or environment. In some embodiments, the wearable device may include earbuds worn in each ear of a user, each earbud including one or more sensors for measuring biometric data related to a user's sleep. The wearable device may interface with and/or communicate with a supplemental device. In some embodiments, the supplemental device may be a charging case that may be capable of charging the earbuds. Biometric data collected using the wearable device may be used to determine sleep information including a user's sleep state and/or a sleep stage. In some embodiments, an audio output of the wearable device may be adjusted to improve a user's sleep conditions. In some embodiments, a beside speaker may be used in addition to or instead of the wearable device to improve a user's sleep.

### REFERENCE TO CROSS-RELATED APPLICATIONS

This application is a continuation of International (PCT) Patent Application No. PCT/US2024/042742, filed Aug. 16, 2024, and titled “Wearable Device for Sleep Applications, and Systems and Methods for Operation Thereof,” which claims priority to U.S. Provisional Patent Application No. 63/533,019, filed Aug. 16, 2023, and titled “Wearable Device for Sleep Applications, and Systems and Methods for Operation Thereof,” the disclosure of each of which is incorporated herein by reference.

### TECHNICAL FIELD

Embodiments described herein relate generally to devices, systems, and methods for sleep applications, and more particularly to devices, systems, and methods for monitoring and analyzing metrics related to a user's sleep and adjusting the user's sleep environment, e.g., to improve quality of sleep of the user.

### BACKGROUND

Sleep plays an integral role in an individual's overall health and quality of life. Studies have shown that sleep deficiency negatively impacts cognitive function, metabolism, and immune function, and may lead to many chronic health conditions including heart disease, high blood pressure, diabetes, depression, and more. Despite this, lack of sleep has become a global problem, with many around the world experiencing nightly sleep disturbances and/or at least one condition that may impact quality of sleep such as insomnia, tinnitus, or obstructive sleep apnea (OSA). The most accurate sleep monitoring systems, such as polysomnograms (PSG), require a user to wear multiple wired sensors on the skin, in the nose, on the finger, and around the chest and abdomen, which may be uncomfortable and/or inconvenient. However, sleep monitoring systems that provide more convenient sleep tracking often lack accuracy and/or detail in the sleep metrics acquired. Additionally, sleep interventions aimed at improving sleep quality typically include supplement use (e.g., Melatonin) or behavioral changes (e.g., turning lights off earlier, limiting device usage, and limiting caffeine and alcohol intake). However, these interventions lack a personalized approach to improving a user's sleep. Therefore, there is a need for devices, systems, and methods that focus on accuracy and comfort when monitoring a user's sleep and that employ a personalized approach to improving a user's sleep quality.

### SUMMARY

Embodiments described herein relate to devices, systems, and methods for monitoring metrics related to a user's sleep and adjusting the user's sleep environment, e.g., to improve quality of sleep of the user.

In some aspects, a wearable device includes a housing configured to be worn in an ear of a user; a sensor disposed on or in the housing, the sensor configured to measure a body metric of the user; a speaker configured to deliver an audio output into an ear of the user; a processor configured to: receive data associated with the body metric from the sensor; and control the speaker to deliver the audio output; and a communication interface configured to send the data associated with the body metric to an external device such that the external device, in response to receiving the data associated with the body metric, is configured to determine a characteristic of a sleep session of the user.

In some aspects, a wearable device includes a processor, a sensor configured to measure a body metric of a user, a speaker configured to transmit an audio output into an ear of the user to assist the user in falling asleep or staying asleep, and a communication interface configured to transfer information bidirectionally with one or more supplementary devices; the one or more supplementary devices configured to determine a sleep stage of the user, and the wearable device configured to be worn in an ear of the user.

In some aspects, a system includes a wearable device configured to be worn in an ear of a user, the wearable device including: a first set of one or more sensors configured to measure a body metric of the user; a speaker configured to deliver an audio output into the ear of the user; and a processor operatively coupled to the sensor and the speaker, the processor configured to receive data from the one or more sensors and to control the speaker to deliver the audio output; and a charging device configured to charge the wearable device when the wearable device is coupled to the charging device, the charging device including a second set of one or more sensors configured to measure an environmental metric of an environment that the user is located in.

In some aspects, a system includes a wearable device configured to be worn in an ear of a user, and a supplementary device including one or more sensors configured to measure one or more metrics from the environment of the user; the wearable device including a processor, a sensor configured to measure a body metric from within the ear of the user, a speaker configured to transmit an audio output into the ear of the user, and a communication interface configured to communicate bidirectionally with the supplementary device.

In some aspects, a method includes measuring one or more body metrics using a sensor of a wearable device that is worn in an ear of a user; determining a sleep stage of the user based on the one or more body metrics; generating an audio output based on the sleep stage of the user; and outputting, via a speaker of the wearable device, the audio output.

In some aspects, a method may include measuring one or more body metrics using a sensor included in a wearable device configured to be worn in an ear of a user; determining a sleep stage of the user based on the body metric measured; and adjusting an audio output from a speaker included in the wearable device based on the sleep stage of the user. In some embodiments, the method may further include calculating a fast motion metric, a slow motion metric, and a respiratory rate of the user using the one or more body metrics measured. In some embodiments, the method may further include displaying the sleep stage of the user on a user device. In some embodiments, the method may further include measuring a metric from an environment in which the user sleeps, and adjusting the audio output of the speaker based on the metric from the environment measured.

### BRIEF DESCRIPTION OF THE DRAWINGS

FIG. 1 is a schematic block diagram of a system used for sleep applications, according to an embodiment.

FIG. 2 schematically depicts a network of devices including systems and devices configured to implement one or more sleep applications, according to an embodiment.

FIGS. 3A-3C schematically depict embodiments of a system used for sleep applications, according to embodiments. FIGS. 3A-3B schematically depict earbuds and a charging case; FIG. 3C schematically depicts a bedside speaker for sleep applications.

FIG. 4 is a flow chart of a method for determining a fast motion metric, a slow motion metric, and a respiration rate using measurements from a system for sleep applications, according to embodiments.

FIGS. 5-9 are schematic block diagrams of embodiments for distribution of computational load across components of a system for sleep applications, according to embodiments.

FIG. 10 is an exploded view of the wearable device for sleep applications, according to an embodiment.

FIG. 11 is an exploded view of a charging case for a wearable device for sleep applications, according to an embodiment.

FIG. 12 depicts a wearable device for sleep applications disposed in a charging case, according to an embodiment.

### DETAILED DESCRIPTION

Embodiments described herein relate to devices, systems, and methods used for sleep applications. In particular, the embodiments described herein relate to a wearable device for monitoring metrics related to a user's sleep and/or adjusting the user's sleep conditions or environment, and methods and systems for operation thereof.

Humans require sleep to survive, and sleep deficiency can have detrimental effects on many aspects of human health including cognitive function, metabolism, immune function, and much more. Monitoring the sleep of an individual can be challenging due to the trade-off between accuracy and comfort of the monitoring system. A sleep monitoring system should be comfortable so as to not interfere further with a user's sleep; however, the system should also be accurate to properly inform the user about his or her sleep. Each individual has a unique circadian rhythm, sleep schedule, and sleep environment, highlighting the need for a personalized approach to improving sleep quality. Current sleep interventions lack personalization and are incapable of adjusting a user's sleep conditions continuously throughout the night. The embodiments described herein address these drawbacks by implementing both comfortable and accurate monitoring of a user's sleep as well as adjusting the user's sleep conditions to improve the user's quality of sleep.

In some embodiments, the wearable device may include earbuds worn in each ear of the user. Each earbud may include one or more sensors for measuring biometric data related to a user's sleep, a processor, a memory, input/output (I/O) device(s), and communication interface(s). In some embodiments, the wearable device may interface with and/or communicate with a supplemental device. In some embodiments, the supplemental device may be a charging case that may be capable of charging the earbuds. Biometric data collected using the wearable device may be used to determine sleep information of the user including a sleep state (sleep/wake) of the user and/or a sleep stage (i.e., N1 or Stage 1, N2 or Stage 2, N3 or Stage 3, REM) the user is in at a given time point. In some embodiments, the sleep information may be displayed on a user device (e.g., a mobile phone, a tablet, a local computer, a laptop, etc.). In some embodiments, the sleep information and other information collected and/or calculated by the wearable device and/or the supplemental device may be transferred to a server and stored. In some embodiments, the system may include a speaker (e.g., a bedside speaker) or other audio device configured to output audio into an environment in which the user is sleeping. In some embodiments, the speaker or other audio device may include any necessary sensor(s), processor(s), memory, communication interface(s) to monitor and/or adjust the user's sleeping environment. In some embodiments, the speaker may interface to and/or communicate with the wearable device, the supplemental device, or the user device.

Data collection and computational load may be distributed across the devices in the system. In some embodiments, the earbuds may be used to collect raw data and determine body metrics from the raw data; the charging case may be used to analyze the body metrics to determine a sleep state (sleep/awake); the user device may be used to analyze the body metrics to determine a sleep stage and to display sleep information; and a server may be used to store sleep stage history and other information. In some embodiments, the charging case may be used to determine the sleep stage directly, and the user device may be used to display information to the user. In some embodiments, the earbuds may only collect raw data, and the charging case may determine body metrics and then use the body metrics to determine the sleep stage of the user. In some embodiments, body metrics used to determine the sleep stage of the user may include a fast motion metric, a slow motion metric, and/or a respiration rate of the user.

In some embodiments, the earbuds may be configured to output audio directly into the user's ear(s). In some embodiments, the earbuds may output audio that is designed to improve relaxation or to help a user fall asleep (e.g., relaxation content, meditation content, etc.) and/or stay asleep (e.g., masking content such as pink noise). In some embodiments, the earbuds may output audio specific to the user's sleep stage (the sleep stage determined from the body metrics collected by the earbuds). In some embodiments, the audio output may be processed and/or tuned. The computational load of processing and/or tuning the audio output may be distributed across the devices in the system. For instance, in some embodiments, the user device (e.g., a mobile phone, tablet, etc.) may process and/or tune the audio before passing the audio output information through the supplemental device(s) to the wearable device. The wearable device may further process and/or tune (e.g., bass boost, reduce/increase volume) the audio output. In some embodiments, the wearable device may process and/or tune the audio output according to sensor data collected by the system. In some embodiments, both the supplemental device(s) and the wearable device(s) may process and/or tune the audio output (e.g., bass boost, reduce/increase volume) based on sensor data collected by the system.

In some embodiments, the supplemental device(s) may collect data related to the environment in which the user is sleeping and determine an environmental score, the environmental score indicative of how conducive the environment is to sleep. In some embodiments, the audio output of the wearable device may be adjusted according to the environmental score (e.g., increasing volume of calming noises, outputting a particular sound frequency, etc.) to improve a user's sleep conditions.

The system may be used for interventions to help treat medical conditions including insomnia, tinnitus, sleep apnea, etc. In some embodiments, the wearable device may be used to detect body metrics related to breathing and/or snoring during sleep to detect likelihood a user is experiencing obstructive sleep apnea (OSA). In some embodiments, the wearable device may be used to improve tinnitus symptoms through brain training using the wearable device.

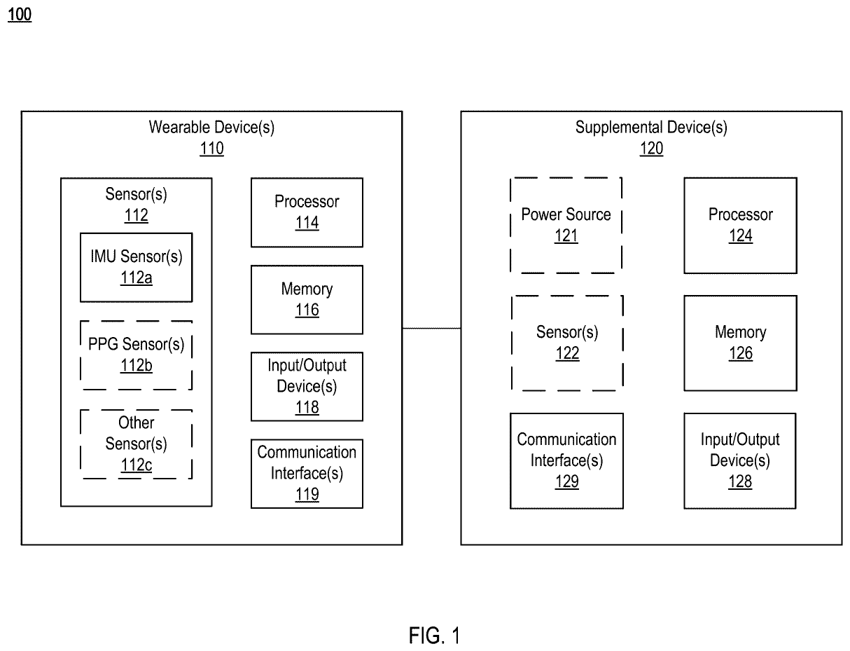

FIG. 1 is a schematic block diagram of a system 100 used for sleep applications, according to an embodiment. As shown, the system 100 includes one or more wearable devices 110 and one or more supplemental devices 120. The wearable device(s) 110 may have sensors 112 including one or more inertial measurement unit (IMU) sensor(s) 112a, and may optionally include one or more photoplethysmography (PPG) sensor(s) 112b and one or more other sensor(s) 112c. The wearable device(s) 110 may include one or more processor(s) 114, a memory 116, one or more I/O device(s) 118, and one or more communication interface(s). In some embodiments, the wearable device(s) 110 may be configured to interface to and/or communicate with the supplemental device(s) 120. The supplemental device(s) 120 may include one or more processor(s) 124, a memory 126, one or more I/O device(s) 128, one or more communication interface(s) 129, and may optionally include a power source 121 and one or more sensor(s) 122.

In some embodiments, the wearable device(s) 110 may include earbuds configured to be worn in a user's ears, and the supplemental device(s) 120 may include a case configured to charge the earbuds. The earbuds may have noise-masking properties to create quieter sleeping conditions for the user. In some embodiments, the structural design of the wearable device(s) 110 may induce passive noise-masking by forming a tight fit with the user's ear. In some embodiments, an audio output from the wearable device(s) 110 may induce active-noise-masking by outputting audio at a louder sound level than that of the ambient environment.

The sensor(s) 112 in the wearable device(s) 110 may be configured to measure physiological or biometric data from the user. For example, in some embodiments, the IMU sensor(s) 112a may be used to measure an acceleration of a portion of the body of the user (e.g., the ear and/or the head) in three dimensions (x direction, y direction, z direction). The PPG sensor(s) 112b may be used to measure change in blood volume in a portion of tissue of the user. In some embodiments, the wearable device 110 includes two earbuds, each earbud including an IMU sensor 112a and/or a PPG sensor 112b such that more than one time series for each biometric may be acquired to improve accuracy of the measurements. In some embodiments, other sensor(s) 112c may be included to enhance measurement of the same biometric data as the IMU sensor(s) 112a and/or the PPG sensor(s) 112b. In some embodiments, other sensor(s) 112c may be included to measure different biometrics. In some embodiments, the other sensor(s) 112c may include a microphone, a body temperature sensor, a pulse oximeter, a galvanic skin response sensor (GSR), one or more electroencephalography (EEG) sensor, etc. In some embodiments, the sensor(s) 112 may be configured to measure biometrics related to whether the user may have abnormal breathing patterns or may be snoring during sleep. In some embodiments, the wearable device(s) 110 may transmit information corresponding to the biometrics measured from the sensor(s) 112 to the supplemental device(s) 120 via the communication interface(s) 119 for further processing. In some embodiments, the wearable device(s) 110 may transmit information corresponding to the biometrics measured to the processor 114 via the communication interface(s) 119 for onboard processing of the biometrics.

The processors 114, 124 can be any suitable processing device(s) configured to run and/or execute a set of instructions or code. For example, the processors 114, 124 can be and/or can include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or the like. The processors 114, 124 can be, for example, a general-purpose processor, central processing unit (CPU), microprocessor, microcontroller, edge AI processor, edge machine learning processor, Field Programmable Gate Array (FPGA), an Application Specific Integrated Circuit (ASIC), a processor board, a virtual processor, and/or the like. The processors 114, 124 can be configured to run and/or execute or implement software application processes and/or other modules, processes and/or functions related to monitoring and improving sleep. The underlying device technologies may be provided in a variety of component types, for example, metal-oxide semiconductor field-effect transistor (MOSFET) technologies like complementary metal-oxide semiconductor (CMOS), bipolar technologies like generative adversarial network (GAN), polymer technologies (e.g., silicon-conjugated polymer and metal-conjugated polymer-metal structures), mixed analog and digital, and/or the like.

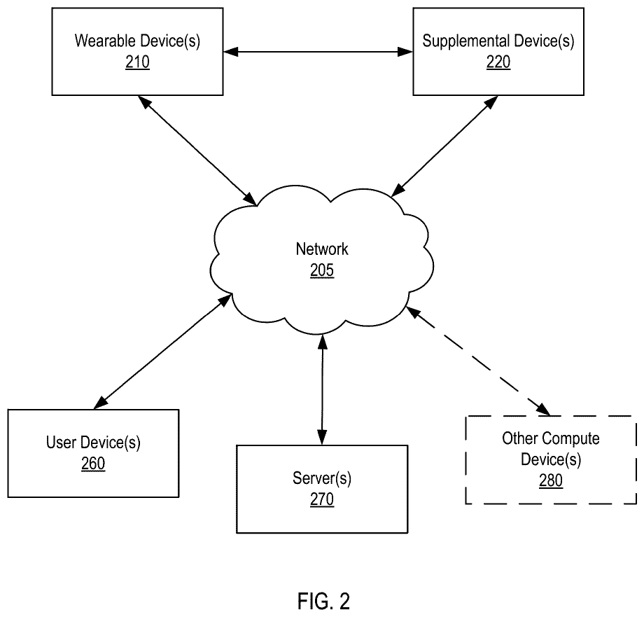

In some embodiments, the processor 114 in the wearable device(s) 110 can be configured to perform any suitable operations for obtaining data collected from the sensor(s) 112; processing and/or analyzing data collected from the sensor(s) 112; and/or sending raw and/or analyzed data to other devices (i.e., the supplemental device(s) 120) via the communication interface(s) 119. Alternatively or additionally, the processor 114 can be configured to send the data from the sensor(s) 112 and/or the memory 116 to one or more external devices or remote devices (e.g., via a network or the cloud) for further processing and/or analysis, described in more detail with respect to FIG. 2. In some embodiments, the processor 114 may be configured to communicate signal(s) to the sensor(s) 112, I/O device(s) 118, communication interface(s) 119, and/or other elements of wearable device(s) 110, to activate and/or control the operation of those elements. For example, in some embodiments, the processor 114 may be configured to perform operations for obtaining audio output information from an external device (e.g., the supplemental device(s) 120), and for transmitting audio output information to the I/O device(s) 118. While not depicted, the wearable device(s) 110 can also include an onboard power supply or be operatively coupled to a power supply that can be configured to supply electrical power to any components of the wearable device(s) 110. In some embodiments, the wearable device(s) 110 can include one or more portable power supplies, e.g., batteries, which can be rechargeable, e.g., via interaction with one or more supplemental device(s) 120 (e.g., a charging case).

The processor 114 may be configured to run an algorithm or operation to analyze raw sensor data acquired by sensor(s) 112 to determine one or more body metrics related to the user's sleep. In some embodiments, the processor 114 may use the acceleration data collected by the IMU sensor(s) 112a to determine a body movement of the user (e.g., a fast motion metric and a slow motion metric) and a respiratory rate of the user. The processor 114 may transmit information relating to the one or more body metrics (e.g., the fast motion metric, the slow motion metric, and the respiratory rate of the user) to the supplemental device(s) 120. The processor 124 in the supplemental device(s) 120 can be configured to perform any suitable operations for obtaining data collected from the sensor(s) 112; analyzing data collected from the sensor(s) 112; and/or sending raw and/or analyzed data to other devices (i.e., a user device or server). In some embodiments, certain aspects of the processor 124 may be functionally and/or structurally similar to the processor 114; therefore, certain aspects of the processor 124 are not described further herein.

In some embodiments, the processor 124 may obtain raw sensor data from the wearable device(s) 110 and run an algorithm or operation to analyze the raw sensor data to determine the one or more body metrics (e.g., the fast motion metric, the slow motion metric, and the respiratory rate of the user). In some embodiments, the processor 124 may directly receive the one or more body metrics from the wearable device(s) and use the one or more body metrics to determine sleep information of the user including a sleep state (sleep/wake) of the user and/or a sleep stage the user is in at a given time point (i.e., N1, N2, N3, REM). In some embodiments, the processor 114 and/or the processor 124 may be configured to analyze raw sensor data to determine whether the user displays abnormal respiration during sleep. For example, in some embodiments, the IMU sensor(s) 112a may transmit acceleration data to the processors 114, 124 and/or a microphone may transmit auditory data to the processors 114, 124. In some embodiments, data from a pulse oximeter sensor in the wearable device(s) 110 may be transmitted to the processors 114, 124. The processor 114 and/or the processor 124 may run an algorithm or operation to use sensor data to detect whether the respiration of the user is abnormal and/or whether the user is snoring to estimate probability the user experiences sleep apnea (or OSA). In some embodiments, the processor 114 and/or the processor 124 may transmit information relating to the user's respiration and/or snoring to the user device (e.g., a mobile phone) to alert the user of possible sleep apnea symptoms.

In some embodiments, the wearable device(s) 110 may store raw sensor data and/or processed data (e.g., determination of body metrics) in the memory 116. The memory 116 on the wearable device(s) 110 can be any suitable memory device(s) configured to store data, information, computer code or instructions (such as those described herein), and/or the like. The memory 116 may store (1) audio output information downloaded from an external device (e.g., the supplemental device(s) 120, the user device, the server etc.); (2) data collected from the sensor(s) 112; (3) and/or any other information related to functioning of the system 100. In some embodiments, the memory 116 can be and/or can include one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some embodiments, the memory 116 can store instructions to cause the processor 114 to execute modules, processes, and/or functions associated with monitoring and improving sleep of a user. In some embodiments, the memory 116 may also be configured to at least temporarily store sensor(s) 112 data, for example, until the data is transmitted to the supplemental device(s), a remote server, or other device (e.g., a user device).

Additionally and/or alternatively, the supplemental device(s) 120 may store raw sensor data (e.g., collected by sensor(s) 112 and/or sensor(s) 122) and processed data in the memory 126. In some embodiments, certain aspects of the memory 126 may be functionally and/or structurally similar to the memory 116; therefore, certain aspects of the processor 126 are not described further herein. The memory 126 may store (1) audio output information downloaded from an external device (e.g., a user device, a server etc.); (2) data collected from the sensor(s) 112, 122; (3) and/or any other information related to functioning of the system 100.

The I/O device(s) 118, 128 may include any suitable device configured to receive input from a user or communicate an output to the user. In some embodiments, the I/O device(s) 118, 128 may include an activation mechanism or a user actuated element (e.g., a touch button, a push button, a switch, etc.) to turn on or activate the wearable device(s) 110 or the supplemental device(s) 120, respectively; or to allow the user to enter information, request information, or initiate or stop audio output. In some embodiments, the I/O device(s) 118, 128 may include a speaker configured to output audio to the user when triggered by a signal from the processors 114, 124 and/or a user input. In some embodiments, the speaker may include, for example, a balanced armature (BA) driver, a dynamic driver, or any suitable transducer to output audio. In some embodiments, the I/O device(s) 118, 128 may include haptics to allow the user to play an audio output, pause an audio output, or move forward/backward through audio outputs. In some embodiments, the speaker may include at least one of a pulse density modulated (PDM) driving circuit or a pulse-code modulation (PCM) (e.g., an inter-integrated circuit sound (I²S)) driving circuit. In some embodiments, the speaker may use both the PDM and the I²S driving circuits. In some embodiments, the use of both PDM and I²S may increase fidelity, reduce artifacts, and/or increase volume of the audio output.

The communication interface(s) 119 can be any suitable device(s) and/or interface(s) that can communicate with the sensor(s) 112, processor 114, memory 116, I/O device(s) 118, or other components of the wearable device(s) 110 and a network (e.g., a local area network (LAN), a wide area network (WAN), or the cloud) and/or an external device (e.g., the supplemental device(s) 120 and/or a user device such as cell phone, tablet, a laptop, or a desktop computer, etc.). Moreover, the communication interface(s) 119 can include one or more wired and/or wireless interfaces, such as, for example, Ethernet interfaces, optical carrier (OC) interfaces, and/or asynchronous transfer mode (ATM) interfaces. In some embodiments, the communication interface(s) 122 can be, for example, a network interface card and/or the like that can include at least an Ethernet port and/or a wireless radio (e.g., a WI-FI® radio, a BLUETOOTH® radio, cellular such as 3G, 4G, 5G, etc., 802.11X Zigbee, etc.). In some embodiments, the communication interface(s) 122 can include one or more satellite, WI-FI, BLUETOOTH, or cellular antenna. In some embodiments, the communication interface(s) 122 can be communicably coupled to an external device (e.g., an external processor) that includes one or more satellite, WI-FI, BLUETOOTH, or cellular antenna, or a power source such as a battery or a solar panel. In some embodiments, the communication interface(s) 119 can be a near-field communication (NFC) device such that information can be exchanged with nearby electronics such as the supplemental device(s) 120. In some embodiments, the communication interface(s) 119 can be BLUETOOTH low energy (BLE) audio. In some embodiments, the wearable device(s) 110 includes a combination of suitable communication interface(s) 119, such as, for example, BLE audio and NFC.

In some embodiments, the communication interface(s) 119 can be configured to receive biometric signals from the sensor(s) 112, processor 114, or other components of the wearable device(s) 110, and to communicate those signals to an external device, e.g., for further processing and/or analysis or presentation to a user. In some embodiments, the communication interface(s) 119 may also be configured to communicate signals to the sensor(s) 112, processor 114, or other components of the wearable device(s), for example, an activation signal to I/O device(s) 118 and/or the sensor(s) 112. In some embodiments, the communication interface(s) 119 transfers information directly to the communication interface(s) 129 in the supplemental device(s) 120. In some embodiments, certain aspects of the communication interface(s) 129 may be functionally and/or structurally similar to the communication interface(s) 119; therefore, certain aspects of the communication interface(s) 129 are not described further herein.

In some embodiments, information related to the functioning of the system 100 (e.g., information related to audio outputs) may be downloaded from the user device and/or the server to the supplemental device(s) 120 and stored in the memory 126 prior to the user going to sleep. The information related to functioning of the system 100 may be transmitted to the wearable device(s) 110 and stored in the memory 116 such that the wearable device(s) 110 may operate while the user is sleeping (or lying down to fall asleep) without connection to any external device. In some embodiments, information stored in memory 126 of the supplemental device(s) 120 may be transmitted to the wearable device(s) 110 via the communication interface(s) 119, 129 without the use of Wifi, 3G, 4G, 5G, or any other form of internet connection. This feature encourages users to disconnect from devices before going to bed, which has been shown to improve quality of sleep. In some embodiments, information relating to audio output can be downloaded from the user device and/or the server directly onto the wearable device(s) 110 and stored in memory 116 prior to the user sleeping (or lying down to fall asleep) such that the wearable device(s) 110 may output audio without connection to the user device or the server. In some embodiments, audio output may be streamed in real-time from the user device or the server such that the wearable device(s) 110 has access to a larger range of audio outputs at a given time rather than only audio outputs stored in the memory 116 and/or 126.

In some embodiments, sleep information of the user may be used to adjust an output of the wearable device(s) 110. In some embodiments, the processor 124 of the supplemental device(s) 120 may transmit a signal associated with the user's sleep state and/or sleep stage to the processor 114 of the wearable device(s) 110 to trigger the wearable device(s) 110 to adjust audio output from the I/O device(s) 118 (e.g., a speaker, a driver, etc.). For example, in some embodiments, the wearable device(s) 110 may be configured to output relaxation content (e.g., nature sounds, meditation, etc.) from the speaker while the user is in the wake state, then automatically transition to masking content (e.g., pink noise, white noise, brown noise) once the user has transitioned into the sleep state to help the user stay asleep. In some embodiments, the wearable device(s) 110 may be configured to output masking content from the speaker while the user is in the wake state and after the user has transitioned into the sleep state. In some embodiments, the wearable device(s) 110 may be configured to produce a “smart alarm” in which the processor 114 may trigger the I/O device(s) 118 (e.g., the speaker, driver, etc.) to automatically transition the audio output to an alarm output when the user transitions into a light sleep stage, such as Stage 1, Stage 2, or REM (as indicated by analysis of the sensor data). A “smart alarm” may wake the user during a light sleep stage such that the user is more rested in the minutes immediately following awakening as compared to being awoken from a deep sleep stage. In some embodiments, the wearable device(s) 110 and/or the supplemental device(s) 120 may be equipped with a real-time clock such that the wearable device(s) 110 and/or supplemental device(s) 120 may output an alarm output at a time chosen by the user without connection to the user device (e.g., the user's mobile phone) or the server. In some embodiments, a mode of the user device may be toggled according to the sleep stage of the user. For example, the user device may be transitioned to “do-not-disturb” or “focus” in response to a signal that the user has entered the sleep state. In some embodiments, the user may input rules or restrictions for interrupting and/or adjusting the mode of the user device (e.g., the user may choose to transition the user device out of “do-not-disturb” upon a call from one or more particular contacts).

In some embodiments, the output(s) of the wearable device(s) 110 and/or supplemental device(s) 120 may be determined by environmental metrics of the environment in which the user is sleeping measured by the sensor(s) 112, 122. In some embodiments, the sensor(s) 112, 122 may sense environmental conditions including, but not limited to, ambient light, ambient noise, humidity, air pressure, and ambient temperature. In some embodiments, the supplemental device(s) 120 may be configured to transmit a signal to control external devices in the environment based on the environmental metrics and/or body metrics measured. For example, the supplemental device(s) 120 may send a signal to toggle a light source in the room “ON” or “OFF” or close blinds in response to the user transitioning into the sleep state (as determined by the wearable device(s) 110 and/or the supplemental device(s) 120). In some embodiments, the supplemental device(s) 120 may send a signal to a thermostat to increase and/or decrease ambient temperature according to the sleep/wake state of the user. For example, the supplemental device(s) 120 may send a signal to the thermostat to decrease ambient temperature in response to the user transitioning into the sleep state and increase the ambient temperature as the user transitions into a light sleep stage and/or prepares to enter the wake state. In some embodiments, the supplemental device(s) 120 may send a signal via a WI-FI network to control external devices connected to the WI-FI network. In some embodiments, the supplemental device(s) 120 may be configured to transmit a signal to control external devices in the environment based on the sleep stage (N1, N2, N3, REM) of the user. In some embodiments, the supplemental device(s) 120 may be configured to determine an environmental score, the environmental score indicative of how conducive the user's environment is to sleep. In some embodiments, the supplemental device(s) 120 may transmit a signal to control the wearable device(s) and/or external devices according to the environmental score (e.g., decreasing thermostat temperature, increasing volume of calming noises from the wearable device(s) outputting a particular sound frequency from the wearable device(s), etc.) to improve a user's sleep conditions. In some embodiments, the environmental metrics measured and/or the environmental score can be displayed to the user via the user device. In some embodiments, a correlation between one or more environmental metrics measured and one or more sleep metrics measured can be displayed to the user via the user device.

In some embodiments, an audio output from the wearable device(s) 110 and/or supplemental device(s) 120 may be automatically adjusted based on environmental conditions measured. For example, in some embodiments, the audio output from the wearable device(s) 110 and/or the supplemental device(s) 120 may be adjusted as ambient noise changes such that the audio output from the wearable device(s) and/or supplemental device(s) 120 substantially masks the ambient noise. For example, audio output from the wearable device(s) and/or supplemental device(s) 120 may be increased if ambient noise is high, such that the wearable device(s) 110 and/or supplemental device(s) 120 substantially masks ambient noise. In some embodiments, the audio output is maintained at a particular sound level above a sound level of the ambient noise. In some embodiments, the audio output from the wearable device(s) 110 and/or supplemental device(s) 120 may be decreased if an environmental auditory stimulus classified as a “critical” or a “wake” stimulus occurs (e.g., baby crying, smoke/intruder alarms, sunrise, or “wake words” chosen by the user). In some embodiments, the sensor(s) 122 may include a temperature sensor that measures the ambient temperature, which may result in the supplemental device(s) 120 to toggle external devices in the environment “ON” or “OFF” such as an HVAC system, a fan, a heating unit, etc. to improve the user's sleep.

In some embodiments, the system 100 including the wearable device(s) 110 may be used to help treat or mitigate tinnitus symptoms. Tinnitus is a condition in which a subject perceives a ringing or buzzing noise in one or both ears and can be associated with hearing loss. Tinnitus symptoms may be treated with brain training by removing particular frequency/frequencies (i.e., a frequency the subject perceives in the absence of a stimulus) from auditory stimuli presented to the user. Problem frequencies (or frequencies that a subject perceives in the absence of a stimulus) may be determined by a healthcare provider through performing a test known as notching. In some embodiments, the wearable device(s) 110 may aid in tinnitus diagnosis. In some embodiments, the system 100 may be configured to perform an automated notching exam to determine the user's problem frequencies. The wearable device(s) may be configured to output a frequency sweep, in which the wearable device(s) outputs audio of different frequencies. The wearable device(s) 110, the supplemental device(s) 120, or the user device may be configured to receive an input from the user regarding what audio frequencies they can hear or not hear. Providing notching exams via the system 100 would allow users to conveniently perform a notching exam at home, improving accessibility to tinnitus diagnostics.

In some embodiments, the system 100 may be configured to provide therapy to the user based on results from a notching exam (e.g., a notching exam administered by a healthcare provider or a notching exam completed by the system 100). In some embodiments, the results from the notching exam performed by the system 100 may be manually or automatically incorporated into the programing of the wearable device(s) 110 such that the wearable device(s) 110 performs a tinnitus treatment or therapy program. In some embodiments, the results from the notching exam performed by the system 100 may be transferable across multiple devices (e.g., other user devices, third-party devices such as those used by a healthcare professional). In some embodiments, the system 100 may be configured to receive information regarding the user's problem frequencies via a user input. In some embodiments, prescribed frequencies may be input by the user or a third-party individual to program audio output information into firmware on a balanced armature (BA) or driver/audio source of the system 100. In some embodiments, the system 100 may be configured to create a “notch” in the frequency output from the wearable device(s) 110. The system 100 may be configured to adjust audio output from the wearable device(s) 110 by removing the user's problem frequency or frequencies from the audio output. For example, in some embodiments, if the user's tinnitus results in persistent perception of a 10 kHz tone, the wearable device(s) 110 may be configured to remove 10 kHz tones from any audio output produced by the wearable device(s) 110. This feature would reduce brain activity associated with perceiving the 10 kHz tone through lateral inhibition, meaning that neurons that respond to 10 kHz tones would be inhibited due to excitation of neighboring neurons (e.g., neurons that respond to 5 kHz tones or 15 kHz tones).

While systems, devices, and methods are described herein as being used for measuring, processing, and/or analyzing a body metric and/or an environmental metric, it can be appreciated that such systems, devices, and methods can be used to measure, process, and/or analyze any number of body metrics and/or environmental metrics, including a plurality of body metrics and/or a plurality of environmental metrics.

FIG. 2 schematically depicts a network used for sleep applications, according to an embodiment. As shown, the network 205 includes a wearable device(s) 210 and a supplemental device(s) 220. The wearable device(s) 210 can be functionally and/or structurally similar to the wearable device(s) 110, described above with reference to FIG. 1. For example, while not depicted in FIG. 2, the wearable device 210 can include sensor(s), a processor, a memory, I/O device(s), or a communication interface(s), which may be structurally and/or functionally the same as those component(s) of the wearable device 110. The supplemental device(s) 220 can be functionally and/or structurally similar to the supplemental device(s) 120, described above with reference to FIG. 1. For example, while not depicted in FIG. 2, the supplemental device(s) 220 can include sensor(s), a processor, a memory, I/O device(s), a communication interface(s), power source(s), and sensor(s) which may be structurally and/or functionally the same as those component(s) of the supplemental device(s) 120. Therefore, further details of these components will not be described again in reference to FIG. 2.

As shown in FIG. 2, the wearable device 210 (e.g., similar to the wearable device 110) and/or the supplemental device 220 (e.g., similar to the supplemental device 120) can be configured to communicate with compute devices, such as one or more user device(s) 260, one or more servers 270, and/or optionally one or more other device(s) 280 (e.g., a healthcare provider device, a third-party device). The wearable device 210 and/or the supplemental device 220 can be configured to communicate with such compute devices via the network 205. The network 205 can include one or more network(s) that may be any type of network (e.g., a local area network (LAN), a wide area network (WAN), a virtual network, a telecommunications network) implemented as a wired network and/or wireless network and used to operatively couple to any compute device, including wearable device 210, supplemental device(s) 220, user device(s) 260, server 270, database 260, and/or other compute device(s) 280.

In some embodiments, the wearable device 210 can be configured to send data (e.g., body metric data related to sleep collected by sensor(s) in the wearable device 210) to the one or more supplemental device(s) 220, user device(s) 260, server(s) 270, and/or one or other device(s) 280. In some embodiments, the wearable device 210 can include onboard processing (e.g., processor 114) to process sensor data (e.g., filter, convert, etc.) prior to sending the sensor data to the one or more supplemental device(s) 220, user device(s) 260, server(s) 270, and/or one or other device(s) 280. Alternatively or additionally, the wearable device 210 can be configured to send biometric sensor data (raw or processed) to the one or more supplemental device(s) 220, user device(s) 260, server(s) 270, and/or one or other device(s) 280, for such devices to perform further processing and/or analysis of the data. In some embodiments, the wearable device 210 can include a communication interface (e.g., communication interface 119) that is configured to allow one-way or two-way communication with an external device, including, for example the one or more supplemental device(s) 220, user device(s) 260, server(s) 270, and/or one or other device(s) 280. In some embodiments, the wearable device(s) 210 may be configured to receive signals from the supplemental device 220, user device(s) 260, server(s) 270, and/or other device(s) 280. For example, the wearable device(s) 210 may be configured to receive a signal corresponding to an audio output to be delivered by the wearable device(s) 210 or a signal associated with adjusting a volume of the audio output based on environmental factors detected by other compute devices in the system (e.g., ambient light, ambient noise, etc.).

The user device(s) 260 can be compute device(s) that are associated with a user tracking his/her sleep information. Examples of user device(s) 260 can include a mobile phone (or other portable device, such as, for example, a tablet, a laptop, a personal computer, a smart device, etc.). In some embodiments, a user device 260 can be configured to analyze to present (e.g., via a display) the sensor data (raw and/or processed) collected from the wearable device 210.

The server(s) 270 can include compute devices for processing and/or analyzing sleep data, storing sleep information, or storing other information transferred from the wearable device(s) 210, the supplemental device(s) 220, and/or the user device(s) 260. Server(s) 270 can be in a location that is the same as or different from the wearable device(s) 210, the supplemental device(s) 220, and/or the user device(s) 260. For example, server(s) 270 can be located in the same room as the wearable device 210 (e.g., in the user's home). Alternatively, server(s) 270 can be located at a remote location (e.g., such as with a cloud-based server). The server(s) 270 can store information that can be accessible to wearable device 210, user device(s) 250, server(s) 270, and/or other device(s) 280. In some embodiments, server(s) 270 can be a hard drive, a database, a cloud storage, a network-attached storage device, or other data storage device. In some embodiments, server(s) 270 can store sensor data, historical user data including data associated with sleep history, etc.

The other device(s) 280 can be compute device(s) associated with other individuals or entities that have requested and/or been provided access to a user's data such as a healthcare provider, a family member of the user, etc. While not depicted in FIG. 2, it can be appreciated that the compute device 210, the user device(s) 260, the server(s) 270, and/or other device(s) 280 each can include components (e.g., a memory, a processor, a I/O device, etc.) that enable the compute devices to perform functions associated with monitoring sleep of a user and operation of the wearable device 210.

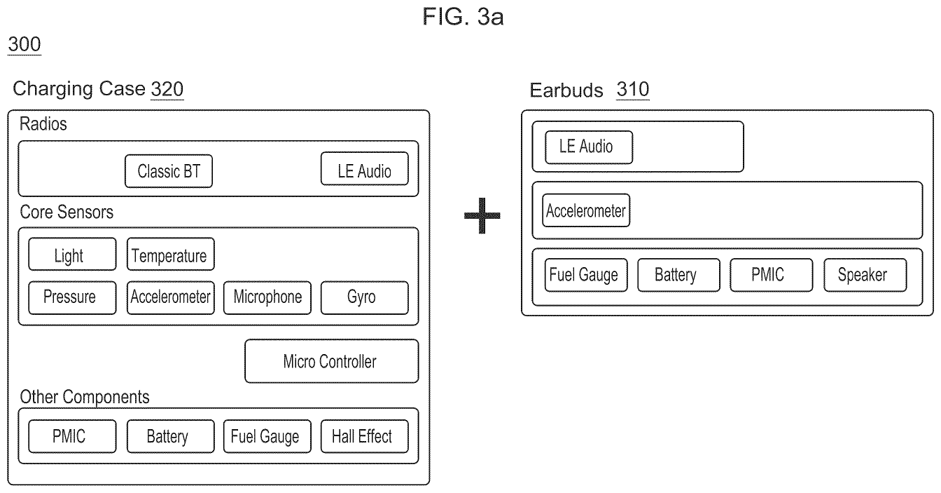

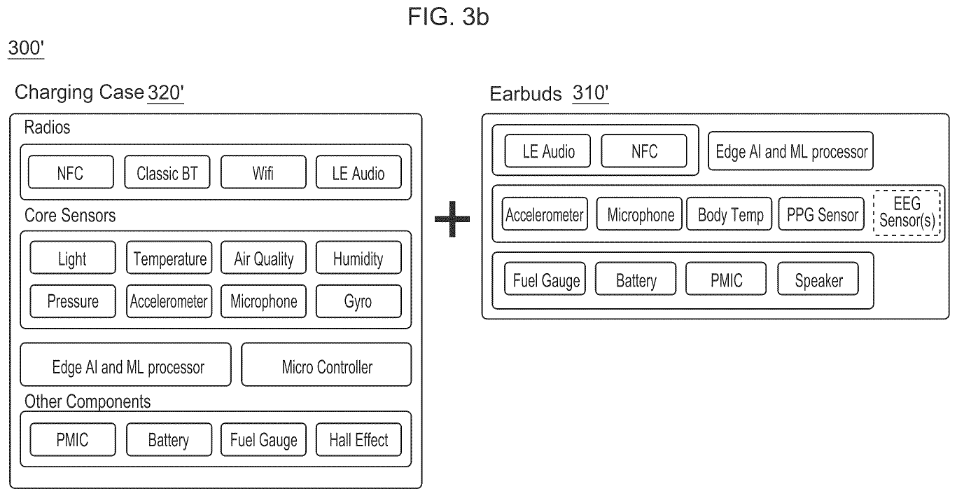

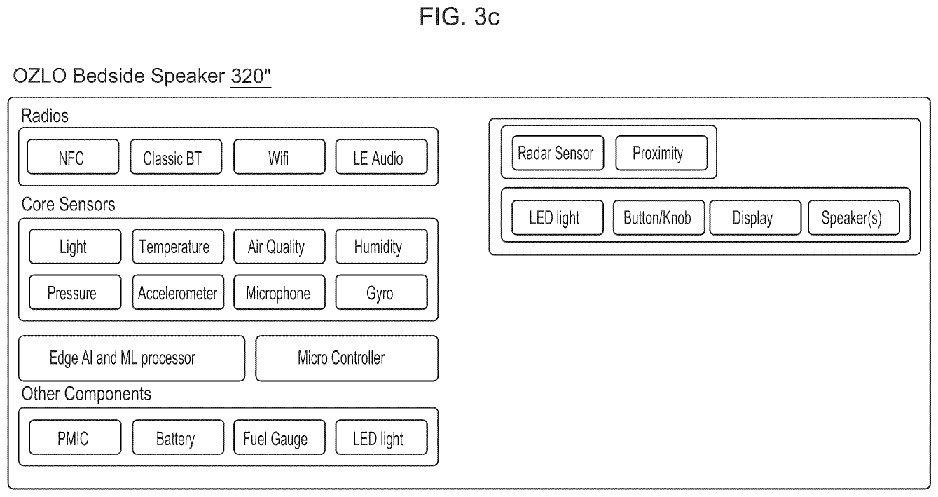

FIG. 3A schematically depicts embodiments of a system for sleep applications 300, according to an embodiment. As shown, the system 300 includes earbuds 310 and a charging case 320. In some embodiments, certain aspects of the earbuds 310 and the charging case 320 may be structurally and/or functionally similar to the wearable device 110 and the supplemental device(s) 120, and therefore certain aspects of the earbuds 310 and the charging case 320 will not be described herein with respect to FIG. 3A. As shown, the charging case 320 may have radios including classic BLUETOOTH and/or BLE audio; core sensors including a light sensor, a temperature sensor, a pressure sensor, an accelerometer, a microphone, and a gyroscope; a processor including a microcontroller; and other components including a power management integrated circuit (PMIC), one or more batteries, a fuel gauge, and a Hall effect sensor. In some embodiments, the fuel gauge may be a metric calculated from other components in the system and displayed to the user. The earbuds 310 may have radios including BLE audio; an accelerometer; and other components including a PMIC, one or more batteries, a fuel gauge, and a speaker. In some embodiments, the fuel gauge may be a metric calculated from other components in the system and displayed to the user. In some embodiments, the earbuds 310 and charging case 320 may transfer information bidirectionally via BLE audio.

In some embodiments, the accelerometer in the earbuds 310 may record acceleration of a portion of the user's body, and this information may be transferred to the charging case 320 and/or another external device (e.g., the user device and/or the server) for processing and analysis to determine the sleep state and/or sleep stage of the user. In some embodiments, information relating to audio output may be transferred or downloaded from the charging case 320, the user device, and/or the server to the earbuds 310 such that the earbuds 310 can output audio to the user via the speaker. In some embodiments, the information relating to audio output may be transferred or downloaded to the charging case 320 prior to use of the earbuds 310 such that the user may listen to audio from the earbuds 310 without connection to the user device, the server, or any other system that may require an internet connection. In some embodiments, the charging case 320 may receive information relating to biometric data from the earbuds 310. In some embodiments, the charging case 320 may obtain information about the user and/or the user's environment via the core sensors (e.g., ambient temperature, ambient light intensity, air pressure, ambient noise level, etc.). In some embodiments the charging case 320 may transfer this information (i.e., biometric data and environmental data) to an external device (e.g., the user device or the server) via classic Bluetooth and/or BLE audio. In some embodiments, the user may control functioning of the earbuds 310 via inputs to the charging case 320. For example, the user may turn the earbuds 310 “ON” or “OFF”, output audio from the earbuds 310, choose type of audio output from the earbuds 310, increase the volume of the audio output from the earbuds 310, initiate sensor recording in the earbuds 310, via the input(s) to the charging case 320.

FIG. 3B schematically depicts a different embodiment of the system for sleep applications 300′. The system 300′ includes earbuds 310′ and a charging case 320′. As shown, the charging case 320′ may have radios including NFC, classic BLUETOOTH, WI-FI, BLE audio; core sensors including a light sensor, a temperature sensor, an air quality sensor, a humidity sensor, a pressure sensor, an accelerometer, a microphone, and a gyroscope; a processor including a microcontroller and an edge AI and ML processor; and other components including a PMIC, one or more batteries, a fuel gauge, and a Hall effect sensor. In some embodiments, the fuel gauge may be a metric calculated from other components in the system and displayed to the user. The earbuds 310′ may have radios including NFC and BLE audio; core sensors including an accelerometer(s), microphone(s), a body temperature sensor(s), PPG sensor(s), and optionally one or more EEG sensors; and other components including a PMIC, one or more batteries, a fuel gauge, and a speaker. In some embodiments, the EEG sensor(s) may be configured to record brain signals from the user. In some embodiments, the brain signals may be processed and/or analyzed to determine information related to the sleep of the user (e.g., a sleep stage, a brain state, etc.) In some embodiments, the fuel gauge may be a metric calculated from other components in the system and displayed to the user. In some embodiments, certain aspects of the earbuds 310′ and charging case 320′ may be structurally and/or functionally similar to the wearable device 110 and earbuds 310 and the supplemental device(s) 120 and charging case 320, and therefore certain aspects of the earbuds 310′ and charging case 320′ will not be described herein with respect to FIG. 3B.

In some embodiments, the core sensors in the earbuds 310′ may collect information about the user. For example, in some embodiments, the accelerometer and PPG sensor(s) in the earbuds 310′ may record data from the user to determine the sleep stage of the user. The microphone may record noise from nearby the user to determine, for example, whether a user is snoring or experiencing other sleep disturbances throughout the night. The user information may be transferred to the charging case 320′ and/or another external device (e.g., a user device and/or server) for processing and analysis. In some embodiments, the user information may be locally processed and analyzed via the onboard edge AI and ML processor, and the processed information may be transferred to the charging case 320′ and/or another external device. In some embodiments, the earbuds 310′ may stream audio output information directly from the user device and/or the server via one of the onboard radios.

FIG. 3C depicts a bedside speaker 320″ for sleep applications, according to an embodiment. As shown, the bedside speaker 320″ may have radios including NFC, Classic Bluetooth, Wifi, and BLE audio; core sensors including a light sensor, a temperature sensor, an air quality sensor, a humidity sensor, a pressure sensor, an accelerometer, a microphone, a gyroscope, a radar sensor, and a proximity sensor; one or more processors including a microcontroller and an edge AI and ML processor; and other components including a PMIC, battery, fuel gauge, LED light(s), button(s) and/or knob(s), display, speaker(s). In some embodiments, the fuel gauge may be a metric calculated from other components in the system and displayed to the user. In some embodiments, the bedside 320″ may be used additionally and/or alternatively to the earbuds and charging case. In some embodiments, the bedside speaker 320″ may collect environmental data via the core sensors as well as output audio via the speaker(s). The bedside speaker 320″ may be useful for users who may not be able to or may not want to sleep with earbuds (e.g., users who prefer not to sleep with a device in their ear).

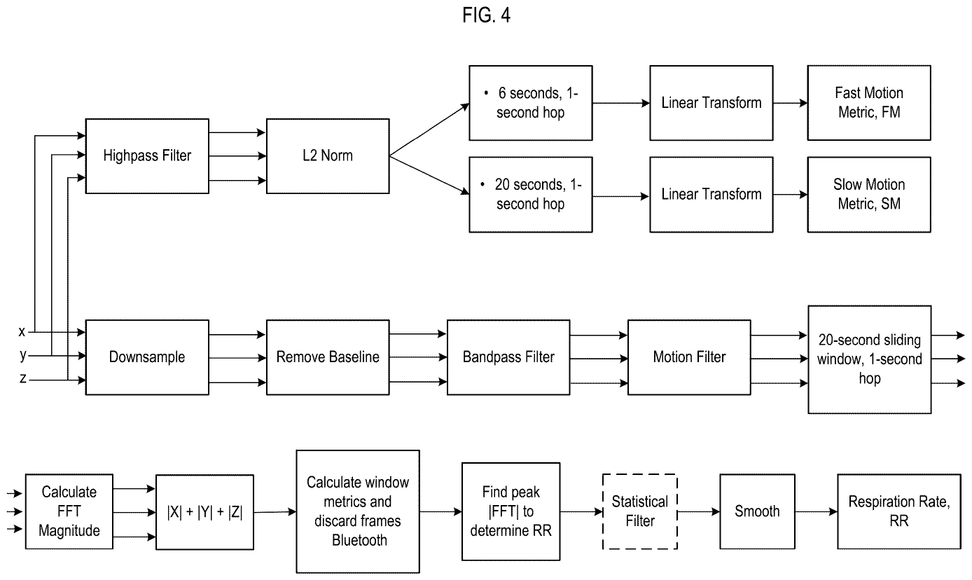

FIG. 4 is a flow chart of a method for determining body metrics of the user that relate to sleep including a fast motion (FM) metric, a slow motion (SM) metric, and a respiration rate (RR) using measurements from the wearable device(s). Acceleration of the wearable device(s), which corresponds to acceleration of a portion of the user's ear, in the “x”, “y”, and “z” direction is collected via the accelerometer in the wearable device. In other words, the accelerometer data may be 3-axis. The acceleration data is sent to a processor (e.g., a processor in the wearable device, the supplemental device, a user device, and/or a server). In some embodiments, the accelerometer data may be 12-bit. In some embodiments, the accelerometer data may be sampled at a rate between about 10 Hz and about 100 Hz, inclusive of all ranges and subranges therebetween. In some embodiments, the accelerometer data may be sampled at a rate of about 25 Hz.

To calculate the fast motion (FM) metric, a high-pass filter is applied to the acceleration data (x direction, y direction, and y direction) then L2 normalization of the filtered data is performed. In some embodiments, the high-pass filter may operate with a cut-off frequency of between about 0.1 Hz and about 1 Hz, including all sub-ranges and values therebetween. Then, the normalized data stream may be broken into sliding time windows (e.g., moving windows). For example, the data stream may be broken into multiple overlapping time windows each with a fixed size of about 6 seconds (s) and being offset from one another by a sliding period of about 1 s. The data points within each time window may be summed. In some embodiments the window size used when calculating the FM metric is in a range of about is to about 1 minute. In some embodiments, the window size used is about 1 s, about 2 s, about 3 s, about 4 s, about 5 s, about 6 s, about 7 s, about 8 s, about 9 s, about 10 s, about 20 s, about 30 s, about 40 s, about 50 s, about 1 minute including all values and subranges therebetween. In some embodiments, the sliding time period used when calculating the FM metric is in a range of about 0.1 s to about 10 s. In some embodiments, the sliding time period used when calculating the FM metric is about 0.1 s, about 0.2 s, about 0.3 s, about 0.4 s, about 0.5 s, about 0.6 s, about 0.7 s, about 0.8 s, about 0.9 s, about is, about 2 s, about 3 s, about 4 s, about 5 s, about 6 s, about 7 s, about 8 s, about 9 s, about 10 s. A linear transform may then be applied to the data, resulting in the FM metric. In some embodiments, the FM metric can be a metric or value that is indicative of a degree of fast motion that a user is engaging in.

To calculate the slow motion (SM) metric, a high-pass filter is applied to the acceleration data (x component, y component, and z component) and then L2 normalization of the filter data is performed. In some embodiments, the high-pass filter may operate with a cut-off frequency of between about 0.1 Hz and about 1 Hz, including all sub-ranges and values therebetween. Then, the normalized data stream may be broken into sliding time windows (e.g., similar to the algorithm to calculate the FM metric). In some embodiments, the time window size used in calculating the SM metric is about 20 s, and the sliding time period used is about 1 s. In some embodiments the window size used when calculating the SM metric is in a range of about is to about 1 minute. In some embodiments, the window size used when calculating the SM metric is about 1 s, about 2 s, about 3 s, about 4 s, about 5 s, about 6 s, about 7 s, about 8 s, about 9 s, about 10 s, about 20 s, about 30 s, about 40 s, about 50 s, about 1 minute including all values and subranges therebetween. In some embodiments, the sliding time period used when calculating the SM metric is in a range of about 0.1 s to about 10 s. In some embodiments, the sliding time period used is about 0.1 s, about 0.2 s, about 0.3 s, about 0.4 s, about 0.5 s, about 0.6 s, about 0.7 s, about 0.8 s, about 0.9 s, about is, about 2 s, about 3 s, about 4 s, about 5 s, about 6 s, about 7 s, about 8 s, about 9 s, about 10 s. A linear transform may then be applied to the data, resulting in the SM metric. In some embodiments, the SM metric can be a metric or value that is indicative of a degree of slow motion that a user is engaging in. The FM and SM metrics may differ from one another based on a size of the window being used to process the data. For example, as described above, the FM metric may be evaluated based on a window size of about 6 seconds, while the SM metric may be evaluated on a window size that is greater than the FM metric (e.g., about 20 seconds).

In some embodiments, the linear transform used to calculate the SM metric is the same as the linear transform used to calculate the FM metric. In some embodiments, the linear transform used to calculate the SM metric is different than the linear transform used to calculate the FM metric. While a FM metric and a SM metric are described herein, it can be appreciated that any type of metric or value can be used to assess a characteristic of a user during rest or sleep. For example, a single metric or value (e.g., a motion metric) can be used to indicate whether a user is engaging in slow motion or fast motion. In some embodiments, the type of motion can also be classified, e.g., as slow, fast, or a particular type of slow movement or fast movement, etc. The motion of the user can be used, e.g., by a processor as described above, to determine a sleep state of the user.

In order to calculate respiratory rate, the acceleration data (x component, y component, and z component) is downsampled. In some embodiments, the acceleration data is downsampled by a factor in the range of about 1 to about 20, including all sub-ranges and values therebetween. Next, the baseline data is removed from the downsampled data, and a bandpass filter is applied. In some embodiments, the bandpass filter can be used to remove data outside of typical respiration rates, e.g., between about 5 and about 35 breaths per minute. Next, a motion filter is applied. Then, the data is further processed using a sliding window (i.e., moving window). In some embodiments, a 20 s sliding window with a is gap portion is used. In some embodiments the sliding window when calculating the respiration rate is in a range of about 1 s to about 1 minute can be used. In some embodiments the sliding window used is about 1 s, about 2 s, about 3 s, about 4 s, about 5 s, about 6 s, about 7 s, about 8 s, about 9 s, about 10 s, about 20 s, about 30 s, about 40 s, about 50 s, about 1 minute including all values and subranges therebetween. In some embodiments, the gap portion used when calculating the respiration rate is in a range of about 0.1 s to about 10 s. In some embodiments, the gap portion used is about 0.1 s, about 0.2 s, about 0.3 s, about 0.4 s, about 0.5 s, about 0.6 s, about 0.7 s, about 0.8 s, about 0.9 s, about 1 s, about 2 s, about 3 s, about 4 s, about 5 s, about 6 s, about 7 s, about 8 s, about 9 s, about 10 s. Next, the fast Fourier transform (FFT) magnitude is calculated, and the magnitude of the x component, the y component, and the z component of the processed data is summed. After summation, window metrics are calculated and certain timepoints are discarded. The peak magnitudes of FFT are located to calculate the respiration rate, and the calculated respiration rate data is then smoothed, resulting in the RR metric. In some embodiments, additional filtering (e.g., statistical filtering) may be applied before the respiration rate data is smoothed. For example, end-of-night statistical filtering may be applied. In some embodiments, the end-of-night statistical filtering may be applied to eliminate outliers prior to smoothing the data.

Once calculated, the FM, SM, and RR metrics may be used in conjunction to determine a sleep state and/or sleep stage of the user. In some embodiments, the body metrics may be calculated continuously such that sleep staging may be determined continuously throughout the night. In such embodiments, portions of acceleration data may be obtained by the processor, and the processor may employ the algorithm on portions of acceleration data that is obtained at a given time. In some embodiments, the movement metrics (e.g., FM and SM) processing may operate at about 25 Hz, and the RR processing may operate at about 2.5 Hz. In some embodiments, the FM, SM, and RR metrics may be calculated and/or reported at a rate of about 0.1 Hz to about 2 Hz, inclusive of all ranges and subranges therebetween. In some embodiments, the FM, SM, and RR metrics may be calculated and/or reported at a rate of about 1 Hz (e.g., once per second). In some embodiments, the FM, SM, and RR metrics may be reported along with a timestamp.

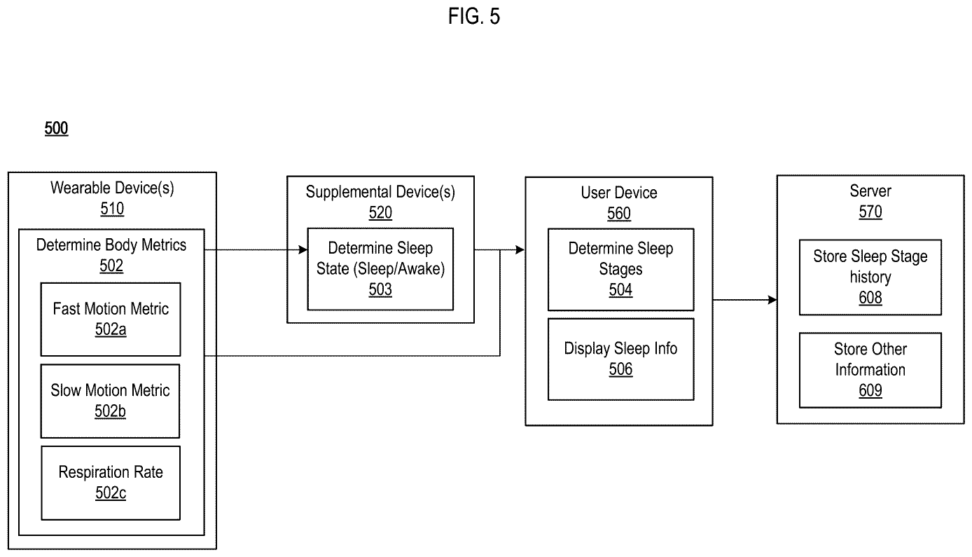

FIGS. 5-9 are schematic block diagrams of embodiments for distribution of functionality and computational load across components of a system for sleep applications. FIG. 5 shows distribution of computational load across a system 500, according to an embodiment. As shown, the computational load is distributed across the wearable device(s) 510, the supplemental device(s) 520, the user device 560, and the server 570. Certain aspects of the wearable device(s) 510, the supplemental device(s) 520, the user device 560, and the server 570 are structurally and/or functionally similar to the wearable device(s) 110, 210, 310, 310′, the supplemental device(s) 120, 220, 320, 320′, 320″, the user device 260, and the server 270; therefore, certain aspects of the wearable device(s) 510, the supplemental device(s) 520, the user device 560, and the server 570 will not be described herein with respect to FIG. 5. As shown, the wearable device(s) 510 is configured to determine body metrics 502 including the FM metric 502a, the SM metric 502b, and RR 502c. The wearable device(s) 510 may include components for determining body metrics 502 such as a processor, a memory, and/or a communication interface. Once the body metrics 502 are determined, this information is transmitted to the supplemental device(s) 520 and/or the user device 560. As shown, the supplemental device(s) 520 utilizes the body metric 502 information to determine a sleep state (sleep/awake) 503 of the user. Information corresponding to the sleep state 503 is then transmitted to the user device 560. The user device 560 may utilize information associated with the body metrics 502, the sleep state 503, or both to determine the sleep stage 504 the user is in as well as display sleep information 506 to the user. The user device 560 may transmit information including but not limited to the body metrics 502, the sleep state 503, and the sleep stages 504 to the server 570. The server 570 may store sleep stage history 508 or store other information 509 collected by the system (e.g., raw sensor data).

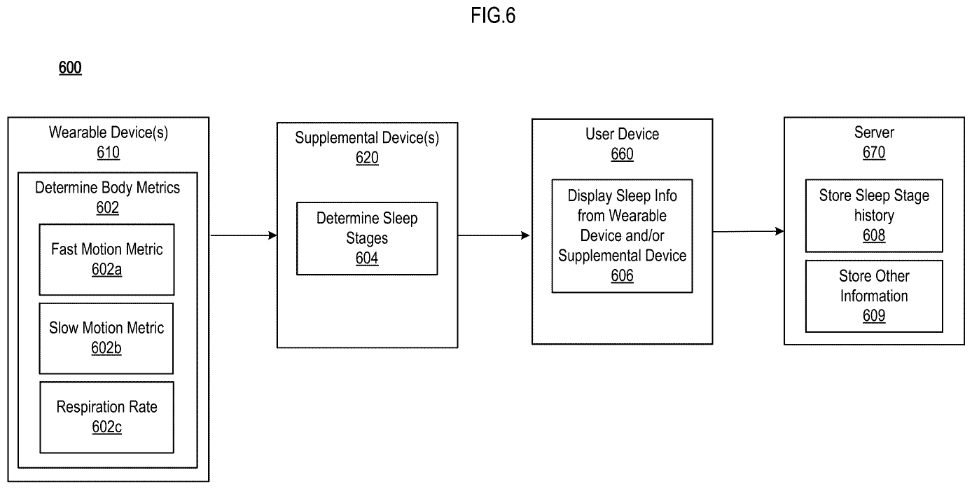

FIG. 6 shows distribution of computational load across a system 600, according to an embodiment. As shown, the computational load is distributed across the wearable device(s) 610, the supplemental device(s) 620, the user device 660, and the server 670. Certain aspects of the wearable device(s) 610, the supplemental device(s) 620, the user device 660, and the server 670 are structurally and/or functionally similar to the wearable device(s) 110, 210, 310, 310′, 510 the supplemental device(s) 120, 220, 320, 320′, 320″, 520, the user device 260, 560, and the server 270, 570; therefore, certain aspects of the wearable device(s) 510, the supplemental device(s) 620, the user device 660, and the server 670 will not be described herein with respect to FIG. 6. As shown, the wearable device(s) 610 is configured to determine body metrics 602 including the FM metric 502a, the SM metric 502b, and RR 502c. Once the body metrics 602 are determined, this information is transmitted to the supplemental device(s) 620. As shown, the supplemental device(s) 620 utilizes the body metric 502 information to directly determine the sleep stages 604 of the user. In some embodiments, the supplemental device(s) 620 may determine the sleep state (sleep/awake) of the user as well as the sleep stages 604 of the user. Information corresponding to the sleep stages 604 is then transmitted to the user device 660. At 606, the user device 660 displays the sleep information from the wearable device(s) 610 (e.g., raw sensor data, biometrics) and/or the supplemental device(s) 620 (e.g., sleep state, sleep stages, environmental data, etc.). The user device 660 may transmit information to the server 670. The server 670 may store sleep stage history 608 or store other information 609 collected by the system (e.g., raw sensor data).

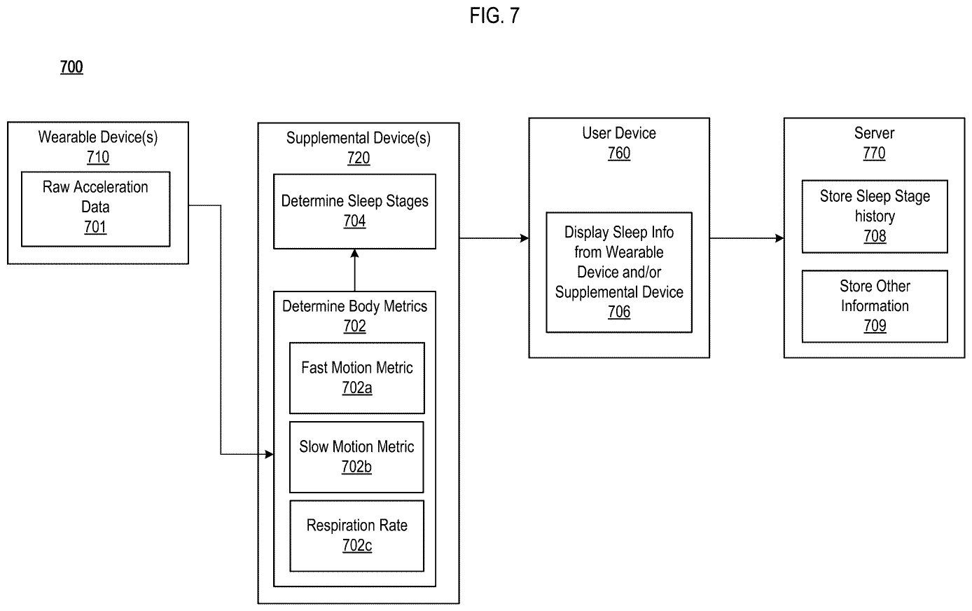

FIG. 7 shows distribution of computational load across a system 700, according to an embodiment. As shown, the computational load is distributed across the wearable device(s) 710, the supplemental device(s) 720, the user device 760, and the server 770. Certain aspects of the wearable device(s) 710, the supplemental device(s) 720, the user device 760, and the server 770 are structurally and/or functionally similar to the wearable device(s) 110, 210, 310, 310′, 510, 610, the supplemental device(s) 120, 220, 320, 320′, 320″, 520, 620, the user device 260, 560, 660, and the server 270, 570, 670; therefore, certain aspects of the wearable device(s) 510, the supplemental device(s) 720, the user device 760, and the server 770 will not be described herein with respect to FIG. 7. As shown, the wearable device(s) 710 is only configured to collect raw acceleration data 701 via sensor(s) in the wearable device(s) 710. Information corresponding to the raw acceleration data 701 is transmitted to the supplemental device(s) 720. The supplemental device(s) 720 is configured to determine the body metrics 702 including FM metric 702a, SM metric 702b, and RR 702c. Once the supplemental device(s) 720 determines the body metrics 702, it utilizes this information to determine the sleep stages 704 of the user. In some embodiments, the supplemental device(s) 720 may determine the sleep state (sleep/awake) of the user as well as the sleep stages 704 of the user. Information determined by the supplemental device(s) is then transmitted to the user device 760. At 706, the user device 760 displays the sleep information from the wearable device(s) 710 (e.g., raw sensor data, biometrics) and/or the supplemental device(s) 720 (e.g., sleep state, sleep stages, environmental data, etc.). The user device 760 may transmit information to the server 770. The server 770 may store sleep stage history 708 or store other information 709 collected by the system (e.g., raw sensor data).

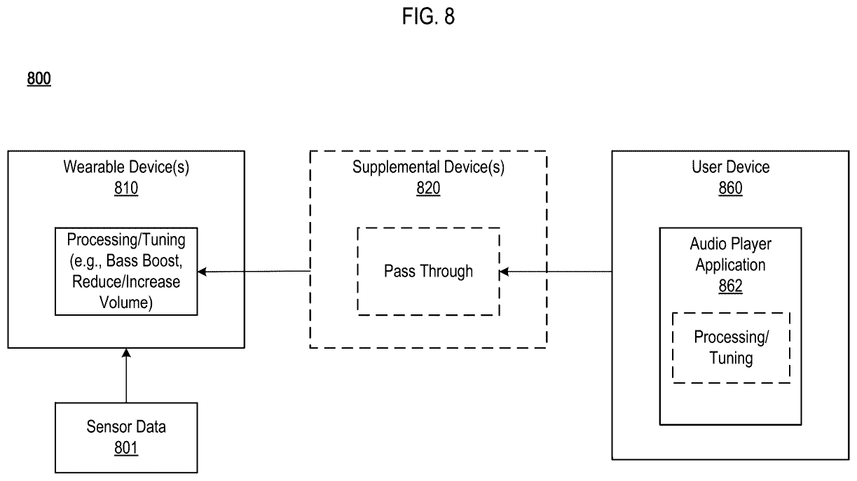

FIG. 8 shows distribution of computational load of processing and/or tuning the audio output across a system 800, according to an embodiment. As shown, the computational load is distributed across the wearable device 810, the user device 860, and optionally the supplemental device(s) 820. In some embodiments, an audio application player 862 executed on the user device 860 may optionally process and/or tune the audio before passing the audio output information through the supplemental device(s) 820 to the wearable device(s) 810. The wearable device(s) 810 may process and/or tune (e.g., bass boost, reduce/increase volume) the audio output prior to its output to the user. In some embodiments, the wearable device(s) 810 may process and/or tune the audio output according to sensor data 801 collected by the system 800. For example, the wearable device(s) 810 may reduce the volume of the audio output in response to a signal that the user has entered the sleep state (e.g., based on biometrics calculated from the sensor data 801).

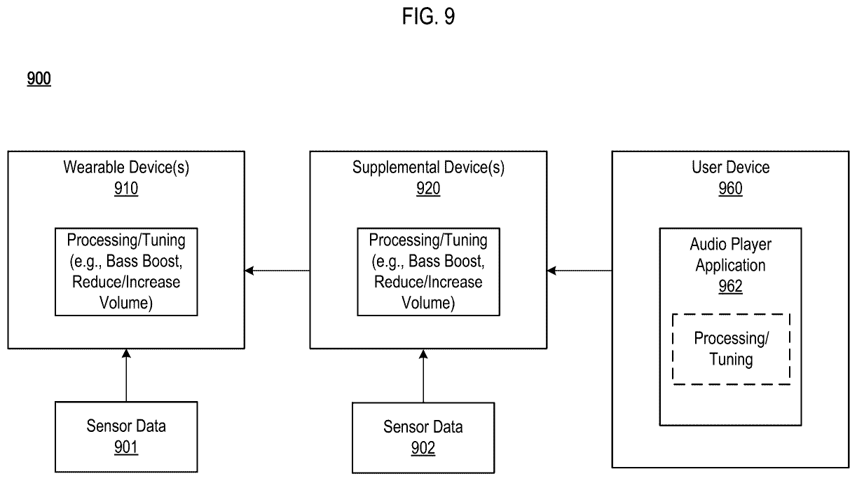

FIG. 9 shows distribution of computational load of processing and/or tuning the audio output across a system 900, according to an embodiment. As shown, the computational load is distributed across the wearable device 910, the supplemental device(s) 920. and the user device 960. In some embodiments, an audio application player 962 executed on the user device 960 may optionally process and/or tune the audio before transmitting the audio output information to the supplemental device(s) 920. As shown, the supplemental device(s) 920 may be configured to process and/or tune (e.g., bass boost, reduce/increase volume) the audio. In some embodiments, the supplemental device(s) 920 may process and/or tune the audio output according to sensor data 902 collected by the system 900. For example, the supplemental device(s) 920 may increase volume of the audio output in response to a signal that the ambient noise (collected from a microphone on the supplemental device 920) in the user's bedroom has increased. The wearable device(s) 910 may process and/or tune (e.g., bass boost, reduce/increase volume) the audio output prior to its output to the user. In some embodiments, the wearable device(s) 910 may process and/or tune the audio output according to sensor data 901 collected by the system 900. For example, the wearable device(s) 910 may reduce the volume of the audio output in response to a signal that the user has entered the sleep state (e.g., based on biometrics calculated from the sensor data 901). In some embodiments, the sensor data 901 and the sensor data 902 may include sensor data from both the wearable device(s) 910 and the supplemental device(s) 920.

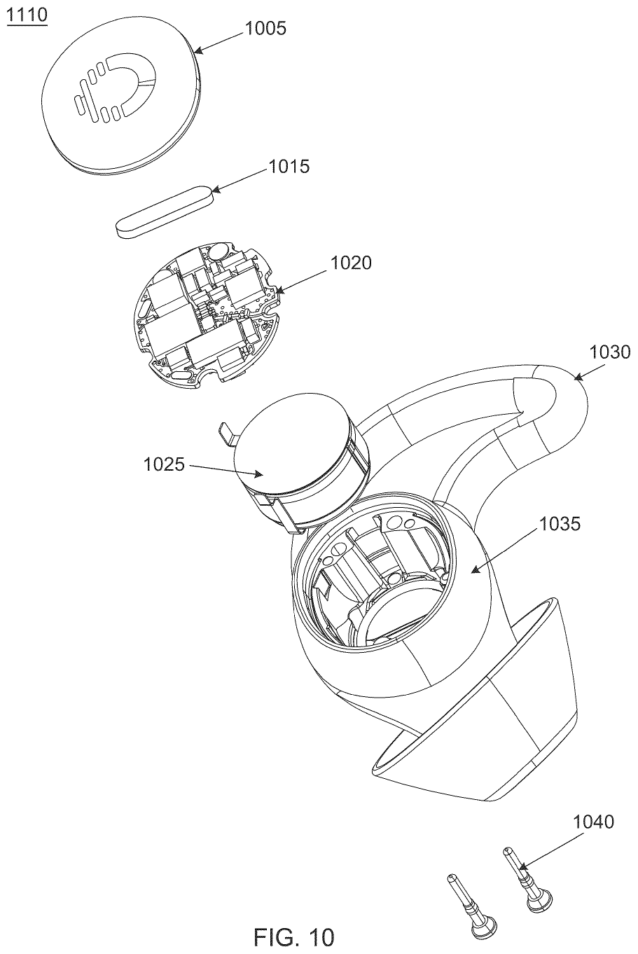

FIG. 10 is an exploded view of the wearable device 1110 for sleep applications, according to an embodiment. As shown, the wearable device 1110 is an earbud configured to be worn in the user's ear. As shown, the wearable device 1110 comprises a main housing 1035 coupled to an eartip (or support member) 1030. The eartip 1030 may be formed in any suitable shape that fits in the user's outer ear. As shown, the eartip 1030 is formed in a curved shape with a point such that the eartip 1030 may fit along the contour of the concha of the outer ear of the user, thereby providing stability of the wearable device 1110 in the user's ear during sleep with little to no discomfort. Additionally, the eartip 1030 may be formed from a flexible material (e.g., silicone, rubber, or any other suitable polymer) such that the eartip 1030 may flex and form comfortably to the user's outer ear. The main housing 1035 may be removably coupled to an earbud tip 1032 (e.g., formed from a material such as silicone, rubber, or any other suitable polymer). In some embodiments, the main housing 1035 may be coupled to different size earbud tips 1032 depending on the anatomy of the user's ear. In some embodiments, the main housing 135, the eartip 1030, and the earbud tip may be configured such that a tight seal between the wearable device 1110 and the user's ear is formed. The tight seal between the wearable device 1110 and the user's ear may provide and/or enhance noise-masking capabilities of the wearable device 1110. In some embodiments, the tight seal between the wearable device 1110 and the user's ear in conjunction with audio output from the wearable device 1110 may provide substantial noise-masking. The tight seal may block or reduce ambient noise that may vibrate anatomy in the inner ear, thereby blocking or reducing an amount of ambient noise perceived by the user. Reducing the amount of ambient noise perceived by the user can improve sleep quality of the user by reducing nightly sleep disturbances caused by ambient noise.

The main housing 1035 defines a compartment in which a battery assembly 1025 and other components of the wearable device 1110 may be disposed. A printed circuit board (PCB) 1020 may be disposed on top of the battery assembly 1025, and a magnet 1015 may be optionally disposed on top of the PCB 1020. A casing 1005 may be coupled to the main housing 1035 to enclose the components of the wearable device 1110 (e.g., the battery assembly 1025, the PCB 1020, the magnet 1015, etc.) therein. The wearable device 1110 may include one or more electrical connector(s) 1040 (e.g., a spring-loaded pin, a pogo pin, etc.) configured to electrically connect the wearable device(s) to a supplemental device (e.g., the charging case). Although shown in a particular form factor, the components of the wearable device 1110 (e.g., the battery assembly 1025, PCB 1020, magnet 1015, connector(s) 1040 etc.) may be arranged in any suitable manner in the main housing 1035.

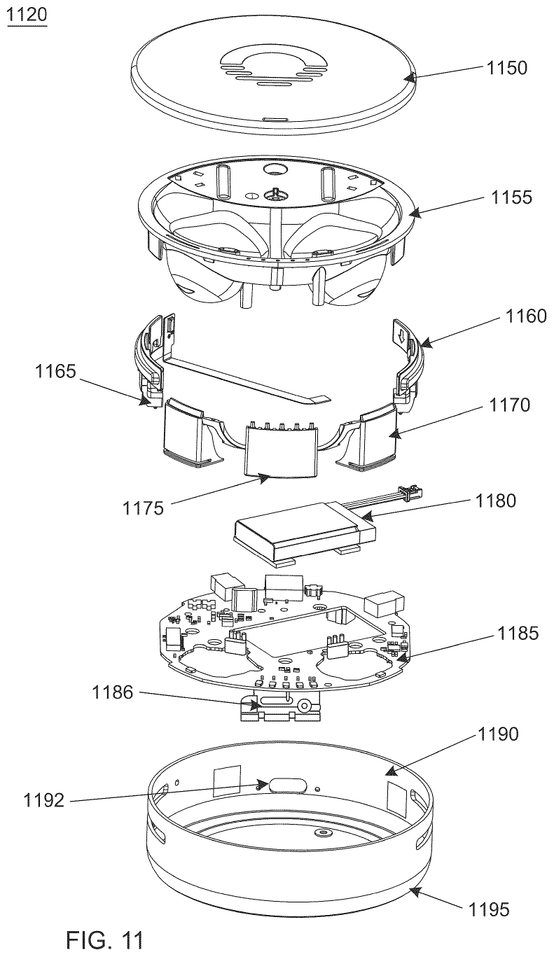

FIG. 11 is an exploded view of a charging case 1120 for the wearable device, according to an embodiment. The charging case may include a base 1195 that supports a bottom portion 1190 of a housing (e.g., a metal can). In some embodiments, the housing may be formed from a strong and rigid material. In some embodiments, the housing can be formed from iron, aluminum, stainless steel, carbon steel, galvanized steel, alloys, plastics, polymers, any other suitable material, or a combination thereof. The bottom portion 1190 of the housing may define one or more openings through which components internal to the housing may be accessible and/or visible from outside of the bottom portion 1190 of the housing. For example, the bottom portion 1190 of the housing may define an opening 1192 through which an LED (not shown) may be visible. In some embodiments, the charging case 1120 may include a conduit (e.g., an LED light pipe, an optical fiber, etc.) (not shown) to transmit light from the LED to the opening defined in the bottom portion 1190 of the housing. The bottom portion 1190 of the housing may define an opening through which one or more sensor(s) included in the charging case 1120 may be exposed. The bottom portion 1190 of the housing may include a sensor cover (not shown) to enclose the opening defining the one or more sensor(s) to protect the one or more sensor(s) from an external environment. The charging case 1120 may optionally include a USB housing (e.g., a USB sheath or USB shroud) 1186. The charging case 1120 may further include a PCB 1185 disposed in the bottom portion 1190 of the housing. A battery 1180 may be electrically connected or coupled to the PCB 1185. An LED housing (e.g., an LED hood) including two lateral components 1170 (a left component and a right component) and a central component 1175 may be disposed in the housing to secure the LED and the conduit in the housing. The charging case 1120 has antenna carriers including a plastic antenna carrier 1165 and a silicone antenna carrier 1160 to secure one or more antennas in the housing 1190.

The bottom portion 1190 of the housing is coupled to a top portion 1155 of the housing, the bottom portion 1190 and the top portion 1155 when coupled forming an enclosed housing. The top portion 1155 of the housing is configured to hold the wearable device(s). In some embodiments, the top portion 1155 of the housing includes one or more (depressions, pits, cavities, dimples, etc.) configured to support the wearable device(s). In some embodiments, the indentations may be formed in a shape corresponding to the wearable device(s) such that the wearable device(s) sit substantially flush with the outer surface of the top portion 1155 the housing. In some embodiments, the top portion 1155 of the housing includes electrical connection points operable to transfer information and/or power to the wearable device(s). For example, the top portion 1155 of the housing may include electrical connection points that may interface with the electrical connector(s) 1040 on the wearable device(s) to charge the wearable device(s). The charging case 1120 may have a lid including an inner lid (not shown) and an outer lid 1150 coupled to the top portion 1155 of the housing. The lid may include a slide guide (not shown) that enables the lid to move from a first position in which the wearable device(s) are enclosed in the housing to a second position in which the wearable device(s) are accessible to the user. Although shown as a sliding lid in FIG. 11, the lid may be coupled to the top portion 1155 of the housing in other ways. For example, in some embodiments, the lid may be coupled to the top portion 1155 of the housing via connectors including, but not limited to, hinges, universal joints, magnets, cantilever joints, torsion snap joints, etc.

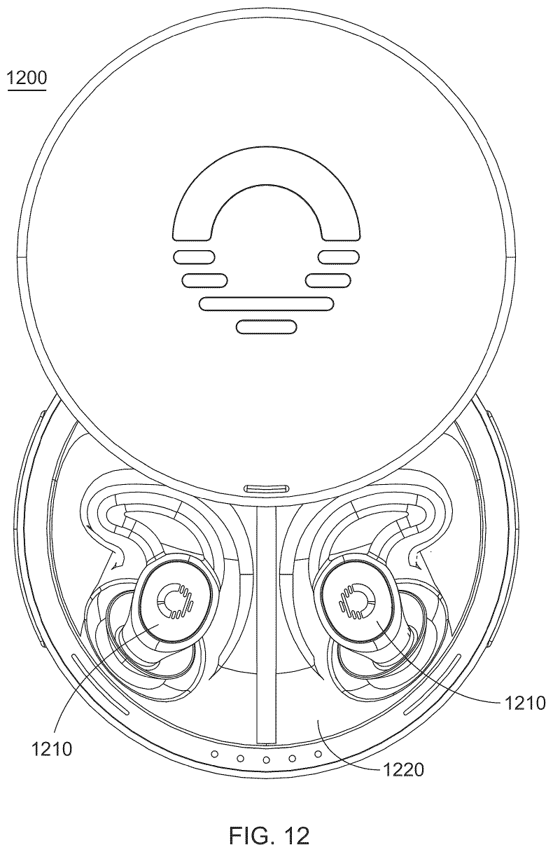

FIG. 12 depicts the wearable device(s) 1210 disposed in the charging case 1220, according to an embodiment. As shown, the wearable device(s) 1210 are earbuds. In some embodiments, the charging case 1220 comprises a housing with indents configured to hold the wearable device(s) 1210. In some embodiments, the charging case 1220 may be configured to charge the wearable device(s) 1210 wirelessly (e.g., via inductive charging). In some embodiments, the charging case 1220 may be configured to charge the wearable device(s) 1210 via electric connection points (e.g., pogo pins, etc.). In some embodiments, the charging case 1220 includes a lid that may be slidably moved or slid to a first position in which the wearable device(s) 1210 are enclosed in the housing to a second position in which the wearable device(s) 1210 are accessible to the user.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Some embodiments and/or methods described herein can be performed by a different software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, lower-level instructions such as assembly code, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

All combinations of the foregoing concepts and additional concepts discussed herein (provided such concepts are not mutually inconsistent) are contemplated as being part of the subject matter disclosed herein. The terminology explicitly employed herein that also can appear in any disclosure incorporated by reference should be accorded a meaning most consistent with the particular concepts disclosed herein.

The terminology used herein is for the purpose of describing particular embodiments only and is not intended to be limiting. As used herein, the singular forms “a”, “an”, and “the” are intended to include the plural forms as well, unless the context clearly indicates otherwise.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

1. A wearable device, comprising:
a housing configured to be worn in an ear of a user;
a sensor disposed on or in the housing, the sensor configured to measure a body metric of the user;
a speaker configured to deliver an audio output into an ear of the user;
a processor configured to:
receive data associated with the body metric from the sensor; and
control the speaker to deliver the audio output; and
a communication interface configured to send the data associated with the body metric to an external device such that the external device, in response to receiving the data associated with the body metric, is configured to determine a characteristic of a sleep session of the user.

2. The wearable device of claim 1, wherein the external device includes a case configured to charge the wearable device.

3. The wearable device of claim 1, wherein the communication interface uses at least one of low energy audio or near-field communication.

4. The wearable device of claim 1, wherein the sensor includes at least one of an accelerometer, a photoplethysmography (PPG), or an electroencephalogram (EEG) sensor.

5. The wearable device of claim 1, wherein the processor is further configured to determine at least one of a fast motion metric, a slow motion metric, or a respiratory rate of the user based on the data associated with the body metric.

6. The wearable device of claim 1, wherein the characteristic of the sleep session of the user includes at least one of: a fast motion metric, a slow motion metric, or a respiratory rate of the user.

7. The wearable device of claim 1, wherein the external device is configured to measure an environmental metric of an environment in which the user is located, and the processor is configured to adjust the audio output delivered by the speaker based on data associated with the environmental metric.

8. The wearable device of claim 1, wherein the processor is configured to adjust the audio output delivered by the speaker based on the data associated with the body metric or the characteristic of the sleep session.

9. The wearable device of claim 1, wherein the processor is further configured to determine a sleep stage of the user based on the data associated with the body metric.

10. A system, comprising:
a wearable device configured to be worn in an ear of a user, the wearable device including:
a first set of one or more sensors configured to measure a body metric of the user;
a speaker configured to deliver an audio output into the ear of the user; and
a processor operatively coupled to the sensor and the speaker, the processor configured to receive data from the one or more sensors and to control the speaker to deliver the audio output; and
a charging device configured to charge the wearable device when the wearable device is coupled to the charging device, the charging device including a second set of one or more sensors configured to measure an environmental metric of an environment that the user is located in.

11. The system of claim 10, wherein the wearable device is an earbud.

12. The system of claim 10, wherein the wearable device further includes a communication interface that is configured to send information to and receive information from the charging device via at least one of low energy audio or near-field communication.

13. The system of claim 10, wherein the first set of sensors includes at least one of an accelerometer, a photoplethysmography (PPG), or an electroencephalogram (EEG) sensor.

14. The system of claim 10, wherein the processor is further configured to determine a fast motion metric, a slow motion metric, or a respiratory rate of the user based on the data from the first set of sensors.

15. The system of claim 10, wherein the charging device is further configured to determine a fast motion metric, a slow motion metric, or a respiratory rate of the user based on the data from the first set of sensors.

16. The system of claim 10, wherein processor is further configured to determine a sleep stage of the user based on the data from the first set of sensors.

17. The system of claim 10, wherein the charging case is further configured to determine a sleep stage of the user based on the data from the first set of sensors.

18. The system of claim 10, wherein the processor is further configured to adjust the audio output based on the data from the first set of sensors, data from the second set of sensors, or a sleep stage of the user.

19. The system of claim 10, wherein the second set of sensors includes at least one of: a light sensor, a temperature sensor, an air quality sensor, a pressure sensor, a humidity sensor, an accelerometer, a speaker, or a gyroscope.

20. A method, comprising:
measuring one or more body metrics using a sensor of a wearable device that is worn in an ear of a user;
determining at least one of a sleep state or a sleep stage of the user based on the one or more body metrics;
generating an audio output based on at least one of the sleep state or the sleep stage of the user; and
outputting, via a speaker of the wearable device, the audio output.
